(19) United States
(12) Patent Application Publication
(10) Pub. No.: US 2022/0146538 A1
(43) Pub. Date: May 12, 2022

(54) METHOD AND DEVICE FOR DETECTING COAGULATION FACTOR INHIBITORS AND ANTIPLATELETS IN A SAMPLE

(71) Applicant: Joseph Louis Spano, San Antonio, TX (US)

(72) Inventor: Joseph Louis Spano, San Antonio, TX (US)

(21) Appl. No.: 17/501,802

(22) Filed: Oct. 14, 2021

Related U.S. Application Data
(60) Provisional application No. 63/113,095, filed on Nov. 12, 2020.

(51) Int. Cl. G01N 33/94; G01N 33/86; G01N 33/543; B01L 3/00

(52) U.S. Cl. CPC G01N 33/94; G01N 33/86; B01L 2300/069; B01L 3/5023; G01N 33/54388

(57) ABSTRACT

Embodiments of the invention provides methods and devices which enable a quick, easy, and cost-effective test for detecting the presence of coagulation factor (CF) inhibitors and platelet (P) inhibitors within small volumes of patient-derived liquid samples. The inventive approach reduces the need for elaborate and time-consuming sample testing or larger quantities of a sample to be collected. The inventive approach overcomes further limitations by providing a detection method that is suitable for measuring antiplatelet drugs, as well as methods for detecting CF inhibitors which are not dependent on competition between CF inhibitors and chromogenic substrates and enzymatic cleaving, but instead relies on non-competitive configurations involving the use of sequential binding moieties with specificity toward CF/P inhibitors and subsequent complexes and/or competitive configurations involving the use of binding moieties that will compete for CF inhibitors against either modified CF inhibitors or other competing moieties.

### CROSS-REFERENCE TO RELATED APPLICATIONS

This application is related to a provisional patent application (Application No. 63/113,095).

### STATEMENT REGARDING FEDERALLY SPONSORED RESEARCH OR DEVELOPMENT

None

### BACKGROUND

### Field of the Art

The disclosed invention generally relates to a method and device for detecting analytes such as coagulation factor inhibitors and antiplatelets in a sample.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

### Discussion of the State of the Art

Current gold standard tests for monitoring anticoagulants (also referred to herein as coagulation factor (CF) inhibitors) are generally focused on assessing clotting activity rather than focusing on more directly detecting and measuring the presence of anticoagulants. Traditional anticoagulants such as Warfarin and heparin require routine monitoring in order to manage clotting disorders and prevent uncontrolled bleeding. Some of the gold standard techniques for monitoring anticoagulants are prothrombin time test with international normalized ratio (PT/INR), activated partial thromboplastin time test (aPTT), plasma-diluted thrombin time test, and thromboelastography. The newer generation of anticoagulants, or direct oral anticoagulants (DOACs), have much more rapid onset and far more predictable pharmacokinetics. For these reasons, it is understood that monitoring of DOAC activity is not required or recommended. However, there are certain instances in the emergency and acute care settings where screening for patients for possible DOAC use could provide vital information for guiding patient management. Unfortunately, the aforementioned gold standard techniques for monitoring clotting activity provide generally inconsistent sensitivity toward DOACs across a range of drug concentrations, and therefore misleading information on coagulation status. This is due to the inherently different modes of action afforded by DOACs from that of Warfarin. It is understood that DOACs have variable effects on the aforementioned routine coagulation assays, depending on the specific drug or drug concentration, the specific assay reagents used for such tests, as well as the patient/indication. Lastly, there are certain commercially available assays, referred to as chromogenic assays, which have reportedly served useful for quantifying one Factor Xa inhibitor (rivaroxaban), but these tests take several hours to perform, are not useful for all DOACs, and they cannot screen or distinguish between individual drugs (clinicians must know the specific drug present in a given patient).

One example method of detecting coagulation factor (e.g. thrombin, factor Xa) inhibitors in a sample involve mixing a sample containing a CF inhibitor(s) with a composition containing a chromogenic substrate conjugated to a detectable substance under conditions which allow the CF to release a detectable substance from the chromogenic substrate. This can be accomplished by adding a test sample containing a CF inhibitor into a matrix (e.g. a reaction mixture or solid support) into which a composition containing a chromogenic substrate conjugated to a detectable substance is added followed by measuring the amount of released detectable substance. The chromogenic substrate will compete against CF inhibitor(s) in the sample for binding with proteolytically active CF. If the amount of CF inhibitor in the sample is low or absent, CF will freely bind to and proteolytically cleave the chromogenic substrate thereby releasing the detectable substance such as a dye. Alternatively, if the amount of CF inhibitor in the sample is high enough, the CF inhibitor will out-compete the chromogenic substrate and bind irreversibly to the CF, thus inhibiting it and preventing it from binding the substrate at its active site, therefore little to no substrate is cleaved and no detectable substance is released. The interaction between the CF, CF inhibitors, and the chromogenic substrates, along with enzymatic cleaving of the chromogenic substrate is critical for this approach to succeed. Furthermore, these approaches present limitations as they cannot differentiate between different CF inhibitors or detect additional CF inhibitors. Additionally, these strategies are not feasible for the detection of additional anticoagulant drugs including at least antiplatelet drugs and COX inhibitors. Thus, no technology currently exists that is capable of directly detecting the presence of CF and platelet inhibitors (also referred to herein as CF/P inhibitors).

### SUMMARY

The present invention overcomes these limitations by providing a detection method that is suitable for measuring antiplatelet drugs, as well as methods for detecting CF inhibitors which are not dependent on competition between CF inhibitors and chromogenic substrates and enzymatic cleaving, but instead relies on non-competitive configuration involving the use of sequential binding moieties with specificity toward CF/P inhibitors and subsequent complexes and/or competitive configurations involving the use of binding moieties that will compete for CF inhibitors against either modified CF inhibitors or a ‘competing moiety.’ Furthermore, the present invention provides a means for directly detecting, measuring and distinguishing between CF/P inhibitors in the form of a lateral flow assay and no such assay for CF/P inhibitors currently exists.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a process for detecting analytes in a sample in accordance with an exemplary embodiment of the invention.

FIG. 2 illustrates a top view (top) and a side view (bottom) of a test strip for detecting analytes in a sample in accordance with an exemplary embodiment of the present invention.

FIG. 3 illustrates a process for indicating the presence of coagulation factor inhibitors and antiplatelets in a sample

### DETAILED DESCRIPTION

The inventive method and device (hereinafter sometimes referred to more simply as “method” or “device”) described herein provides a quick, easy, and cost-effective test for detecting the presence of CF/P inhibitors in a patient sample. The inventive approach reduces the need for elaborate and time-consuming sample testing or larger quantities of a sample to be collected. Specifically, using a small volume of patient derived sample according to the inventive concept herein, can provide a result indicating the presence, absence, or relative concentration of a number of CF/P inhibitors in a patient sample in a matter of minutes.

The inventive method and device described herein provides various benefits and applications such as: directly measuring or quantifying the presence of anticoagulant drugs in body fluids of patients who are receiving (or are suspected of receiving) anticoagulant therapies, identifying subjects who are at risk for accumulating toxic serum concentrations of anticoagulant therapeutics, identifying subjects who are at risk for developing acute hemorrhage as a result of accumulating toxic dosages of anticoagulant drugs, identifying subjects who may be at risk for developing acute hemorrhage while undergoing surgical intervention as a result of their prescribed anticoagulant therapies, assessing patient's compliance with various anticoagulant medication treatment regimens, evaluation of treatment failure or thrombosis, evaluation of patient groups where treatment failure is likely (i.e. obesity), assessment of drug bio-accumulation or overdose due to renal failure or other causes, assessment of patients suspected of deteriorating renal function, assure absence of said anticoagulant drugs prior to thrombolytic therapy, assure absence of said drugs in patients who require urgent surgery, assessment of patients with uncontrolled or life-threatening bleeding, urgent or non-urgent periprocedural management.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

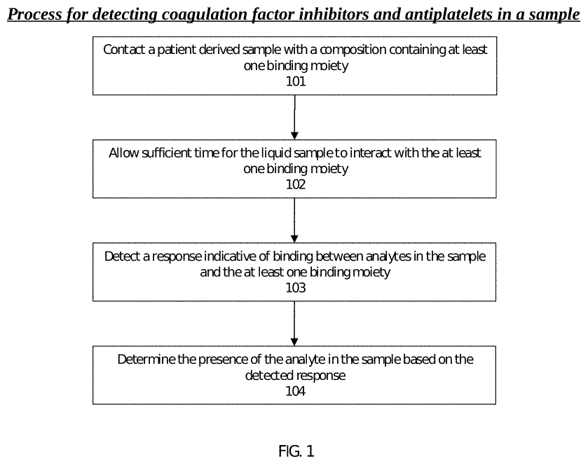

FIG. 1 illustrates an exemplary embodiment of a process for detecting CF/P inhibitors in a sample according to one embodiment. The process includes contacting a patient derived sample with a composition containing at least one binding moiety in step 101, allowing sufficient time for the liquid sample to interact with the at least one binding moiety in step 102, detecting a response indicative of binding between analytes in the sample and at least one binding moiety in step 103, and determining the presence of the analyte in the sample based on the detected response in step 104. The steps may be reorganized or consolidated, as understood by a person of ordinary skill in the art, to arrive at the same end result without departing from the scope of the invention.

In step 101, a patient derived sample is brought into contact with a composition containing at least one binding moiety. The patient derived sample may include patient body fluids such as blood, plasma, serum, urine, or the like or may be a sample derived from patient body fluids. Any patient derived sample may be used without departing from the scope of the invention. The sample may include one or more analytes which the binding moieties may target.

The at least one binding moiety of step 101 includes any moiety capable of targeting and binding with, or competing against, analytes in the sample. These analytes may include one or more CF/P inhibitors, or a metabolite/breakdown byproduct thereof. For example, the binding moieties may include any molecule, or part thereof, capable of binding to, or competing against, CF/P inhibitors and/or their metabolites. Some specific examples include enzymes (e.g. thrombin, factor Xa, COX-2), purified or recombinant CFs, proteins or antibodies (e.g. antithrombin, anti-Xa, anti-CF/P inhibitors), antibody fragments, polypeptides, aptamers, engineered phage (or purified phage tailspike protein), small molecules or other molecular structure capable of binding to, or competing against, CF/P inhibitors or their metabolites. The binding moieties may also compete for one or more analytes against label-conjugated analytes (or analyte analogs) in one example of a competitive assay format. In another example of a competitive assay format, the analytes may compete for binding to a labeled or unlabeled binding moiety (e.g. CF) against a labeled or unlabeled ‘competing moiety’ (e.g. a CF-specific protein such as antithrombin). In another example of a competitive assay, the analytes may compete for binding to a labeled binding moiety against other modified analytes (or analyte analogs). In a different assay format, referred to as a non-competitive (or sandwich) format, the binding moieties may also bind and/or capture a complex comprising analyte and a first binding moiety. For example, in the non-competitive format, a first binding moiety may bind selectively to an analyte forming a first complex, which may be subsequently bound to and/or captured by secondary or tertiary binding moieties.

The CF/P inhibitors, or analytes, that can be detected by the present invention include at least direct oral anticoagulant (DOAC) drugs such as rivaroxaban, apixaban, edoxaban, betrixaban, otamixaban, letaxaban, eribaxaban, fondaparinux, dabigatran, argatroban, inogatran, ximelagatran hirudin, lepirudin, desirudin and/or bivalirudin, antiplatelet drugs such as clopidogrel, ticagrelor, and prasugrel, and COX-2 inhibitors such as acetylsalicylic acid, ibuprofen, celecoxib, difenac, etoricoxib, lumiracoxib, and naproxen. This list is not meant to be limiting, and other CF/P inhibitors could be detected by the same process described herein without departing from the scope of the invention. Furthermore, the detection may also involve detection of a metabolite or breakdown byproduct of any of the listed or other CF/P inhibitors.

At step 102, the sample is given sufficient time to interact with the at least one binding moiety. This duration may be on the order of minutes (e.g. 5-10 minutes), however may be longer or shorter depending on the particular configuration being used. During this time, the one or more analytes in the sample may interact with the at least one binding moiety until a signal is produced by a label associated with the binding process. Suitable labels for this process include at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal.

At step 103, the signal produced by the label is detected indicating binding interaction between analytes in the sample and at least one binding moiety. This detection may be done by visual observation or alternatively via an optical reader or system comprising a photodetector and/or excitation source for detecting the generated signals. In the latter scenario, the optical reader or photodetector configurations may be used to quantify the amount of signal generated.

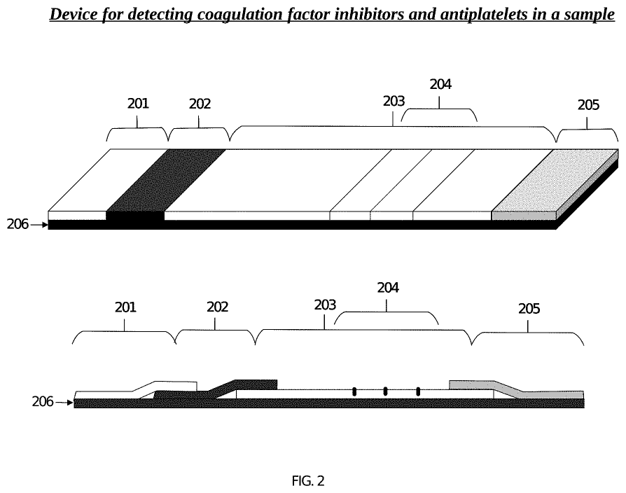

At step 104, the presence of an analyte in the sample is determined based on the detected response. The amount of signal detected is directly related to the amount of analyte present in the sample and is dependent on the configuration of the test being used. For example, in one configuration, if little or no signal is produced and detected, this indicates that little or no analyte is present in the sample. On the other hand, as the amount of signal being produced and detected increases, this indicates higher amounts of analyte present in the sample. In addition, depending on the strength of the signal detected in step 103, it is possible to estimate an amount of analyte present in the sample. Alternatively, the test may be configured in different format such that the absence of signal or a lower detected signal is indicative of higher amounts of analyte present in the sample. These different configurations and outcomes will be discussed in more detail below with respect to FIG. 2.

FIG. 2 illustrate an exemplary embodiment of a test strip for performing the method of FIG. 1. The test strip includes a backing 206 on which is located a sample application pad 201, a conjugate release pad 202, a test region 203 comprising one or more test lines at the analytical region 204, and a wick pad or absorbing pad 205. This test strip may be referred to as a lateral flow assay test strip.

The sample application pad 201 is where the patient derived sample is received. A sufficient amount of sample to flow along the length of test strip should be received at this location. The patient derived sample will gradually flow into the conjugate release pad 202.

The conjugate release pad 202 includes temporarily immobilized and labeled components, which in one embodiment may comprise one or more binding moieties (or one or more sequential binding moieties) for one or more CF/P inhibitors as discussed above in a non-competitive assay. In some embodiments, particularly in a competitive assay, the conjugate release pad may include modified analytes comprising one or more labeled CF/P inhibitors or drug/drug analog(s). In another embodiment of a competitive assay, the conjugate release pad may include temporarily immobilized labeled ‘competing moiety’ which is capable of binding selectively with high affinity to an analyte-specific binding moiety but of substantially lower or no affinity to free analyte or analyte+binding moiety. In another embodiment of a competitive assay, the conjugate release pad may include one or more labeled binding moieties capable of binding selectively with high affinity to CF/P inhibitors which may be present in a sample. As the sample flows from the sample pad 201 to the conjugate release pad 202, the labeled species within the conjugate release pad 202 become mobilized and will bind to, or compete against, CF/P inhibitors present in the sample as flow continues laterally (or vertically in alternative device formats) to the test region 203.

The test region 203 allows for continued lateral (or vertical) flow of the sample which now includes a mobilized labeled species (comprising either labeled binding moieties, labeled ‘competing moiety,’ or modified analytes) released from conjugate release pad 202. In some embodiments, the test region is comprised of a nitrocellulose membrane or other porous material with high protein affinity allowing for immobilization of one or more components at the analytical region 204. Such components may include proteinaceous capture moiety, proteinacious binding moiety, and/or modified analytes/analyte analogs which may be further modified/attached to a protein (e.g. albumin) to facilitate their immobilization to the test region. In one example, which may be in either a competitive or non-competitive format, said labeled binding moieties may be flowing bound or unbound to CF/P inhibitor depending on the amount of CF/P inhibitor present in the sample. Similarly, CF/P inhibitor may be flowing in the sample labeled and unlabeled through the test region depending on the quantity of CF/P inhibitor present in the sample. In one example of a competitive assay, said labeled analytes or labeled ‘competing moieties’ will flow unbound in a mixture containing varying amounts of CF/P inhibitor that may also be present. Flow continues laterally (or vertically in an alternative device format) along test region 203 to the analytical region 204.

The analytical region 204 includes test lines comprising immobilized capture moieties and one or more downstream control line(s). In one embodiment these test lines may be configured in what may be referred to as a sandwich (or non-competitive) format lateral flow assay. In this configuration, these immobilized capture moieties are unbound and will specifically target a first or second complex. As the first complex (comprising CF/P inhibitor+first binding moiety), or alternatively a second complex (first complex +targeting moiety for first complex) in the sample flows by these lines the immobilized capture moieties will bind to the first and/or second complex, capturing them in place. Because the CF/P inhibitor has previously bound to a binding moiety to form at least one labeled complex, when passing through conjugate release pad 202, the CF/P inhibitor, and optionally the second complex, becomes “sandwiched” between the first binding moiety and the capture moiety at a test line leading to a collection of labeled ‘sandwich’ complexes accumulating at a test line. This accumulation of labels at a test line allows for a signal to be produced and detected at a test line which can be used to determine the presence and quantity of analyte present in the sample. In some embodiments, one or more control lines may be present at the analytical region 204 and they may comprise a positive and/or negative control line. The control line(s) serve as a means of validating that the test has successfully run to completion and whether the result(s) indicated at the test line(s) can be trusted. In some embodiments, the absence of a signal at the control line would indicate an assay that has malfunctioned. The control line may contain one more capture moieties specific to any labeled species released from the conjugate pad 202 which have flowed past the test line(s). Accumulation of label at the control line(s) leads to detectable signal along this line and which indicates that the assay is functioning properly.

In an alternate embodiment, which may be referred to as a competitive format lateral flow assay, the capture moiety(s) at the test line comprise immobilized and unbound analyte-specific binding moieties and will bind a modified (labeled) analyte to generate the maximum signal. In this case, the binding moiety has captured as much labeled analyte as possible because there was no competing analyte present in the patient derived sample. When the patient-derived sample includes a CF/P inhibitor, the CF/P inhibitor will migrate alongside the labeled analyte until reaching the appropriate test line. Once the test line is reached, the CF/P inhibitor and labeled analyte will compete against each other for binding to the immobilized binding moiety. When the patient sample includes a maximum or threshold level of CF/P inhibitor, it will out-compete the labeled analyte and thus all of the labeled analyte will pass by the test line. In this configuration, the lower the signal at the test line the higher the amount of CF/P inhibitor present in the sample, because the labeled CF/P inhibitors pass by the test line due to being outnumbered and out-competed by the CF/P inhibitor present in the patient sample. Similarly, a greater signal at the test line indicates lower amounts of CF/P inhibitor present in the sample. The control line(s) in this format may also be present, and may function similarly as described previously.

In another example of a competitive assay format, the test line comprises immobilized unbound ‘competing moiety’ or alternatively a binding moiety. In the former example, the competing moiety (e.g. a CF-specific protein such as antithrombin, or an antibody) will bind exclusively to a labeled analyte-specific binding moiety (e.g. purified CF) released from the conjugate pad and will compete against the analytes for the binding moiety(s). When analyte is present in the patient sample, it will compete against the ‘competing moiety’ for binding to a labeled binding moiety. Once enough analyte is present in the patient sample, it will out-compete the ‘competing moiety’. Thus, a lesser amount of labeled binding moiety will be captured by the ‘competing moiety’ immobilized on the test line. Alternatively, in the latter example the orientation of the ‘competing moiety’ and binding moiety are switched. In other words, now the competing moiety is labeled and it is located upstream in the conjugate release pad, while the binding moiety is unlabeled and it is immobilized on a test line. The same competitive conditions otherwise apply. Likewise, the control line(s) in this format may also be present, and may function similarly as described previously.

In another example of a competitive assay format, the test line comprises immobilized unlabeled analyte(s) or analyte analog(s). In this example, the immobilized analyte will bind to a labeled binding moiety that was released from the conjugate release pad and will compete against other analytes that may be present in the patient sample. When no analytes are present in the sample, the immobilized analyte will capture as much labeled binding moiety as possible and will produce the maximum signal. In this case, the immobilized analyte has captured as much labeled binding moiety as possible because there was no competing analyte (CF/P inhibitor) present in the patient derived sample. When the sample includes a CF/P inhibitor, the CF/P inhibitor will bind to the labeled binding moiety (released from the conjugate release pad) and will migrate to the test line. Once the test line is reached, the CF/P inhibitor originating from the patient-derived sample will compete with the immobilized analyte for binding to the mobilized labeled binding moiety. When the patient sample includes a maximum or threshold level of CF/P inhibitor, it will out-compete the immobilized analyte and thus all of the labeled binding moiety will pass by the test line. In this configuration, the lower the signal at the test line, the higher the amount of CF/P inhibitor present in the sample, because the labeled binding moieties pass by the test line. Similarly, a greater signal at the test line indicates lower amounts of CF/P inhibitor present in the sample. The control line(s) in this format may also be present, and may function similarly as described previously.

In any of the above configurations, the device of FIG. 2 may be configured to detect a single CF/P inhibitor, to generally detect a variety of CF/P inhibitors of the same or similar class, or may be configured to differentiate between various CF/P inhibitors. For example, the device may include a test line at the analytical region 204 specific to a single CF/P inhibitor or generic to a group of CF/P inhibitors. In these scenarios, the binding moieties, modified analyte, or ‘competing moiety’ used in the conjugate pad 202 and/or at the test lines will bind specifically to, or compete against, a specific CF/P inhibitor or to a group of CF/P inhibitors which share a common binding entity or epitope.

As another example, for differentiating between different CF/P inhibitors, the device may include a plurality (two or more) test lines at the analytical region 204 each configured for a different CF/P inhibitor, thereby allowing not only the detection and measurement of a CF/P inhibitor, but particularly indicating which CF/P inhibitor(s) is/are present in the sample in a single test. In this example, the conjugate release pad 202 may include a plurality of different labeled binding moieties, ‘competing moiety,’ or modified analyte(s) and similarly each test line may include a plurality of appropriate binding moieties, ‘competing moiety,’ and/or modified analyte(s) that may specifically bind to, or compete against, different CF/P inhibitors. Accumulation of labeled binding moieties and thus detectable signal at a given test line would indicate the presence of a particular CF/P inhibitor under the sandwich lateral flow assay configuration, while the lack of accumulation and detectable signal at other CF/P inhibitor specific test lines would indicate those CF/P inhibitors are not present or are present at lower levels. Alternatively, under the competitive format lateral flow assay, detectable signal at each CF/P inhibitor specific test line would indicate that CF/P inhibitor is not present or present at lower levels, while a lack of detectable signal at CF/P inhibitor specific test lines would indicate the presence or relative abundance of that particular CF/P inhibitor.

A cassette or housing (not shown) may be used to contain the test strip(s) and provide a viewing window to facilitate investigation of the result displayed at the analytical region of the test strip, which may be performed visually or quantitatively via the use of a suitable reader technology.

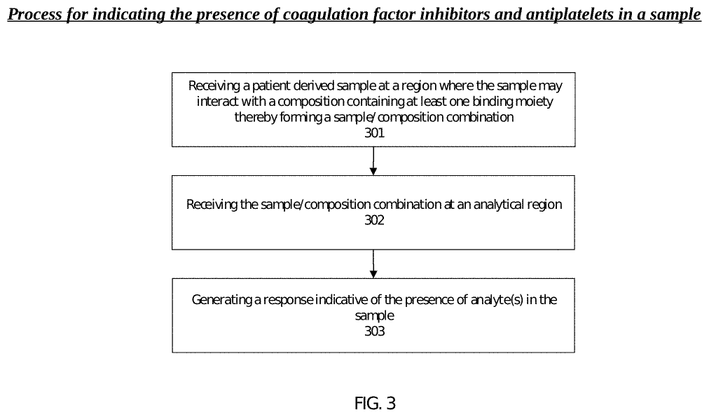

FIG. 3 illustrates an exemplary embodiment of a process for indicating the presence of CF/P inhibitors in a sample according to one embodiment. The process includes receiving a patient derived sample at a region where the sample may interact with a composition containing at least one binding moiety thereby forming a sample/composition combination in step 301, receiving the sample/composition combination at an analytical region in step 302, generating a response indicative of the presence of analyte(s) in the sample at step 303. The steps may be reorganized or consolidated, as understood by a person of ordinary skill in the art, to arrive at the same end result without departing from the scope of the invention.

In step 301, a patient derived sample is received at a region comprising a composition containing at least one binding moiety. The patient derived sample may include patient body fluids such as blood, plasma, serum, urine, or the like or may be a sample derived from patient body fluids. Any patient derived sample may be used without departing from the scope of the invention. The sample may include one or more analytes which the binding moieties may target.

The at least one binding moiety of step 301 includes any moiety capable of targeting and binding with, or competing against, analytes in the sample. These analytes may include one or more CF/P inhibitors, or a metabolite/breakdown byproduct thereof. For example, the binding moieties may include any molecule, or part thereof, capable of binding to, or competing against, CF/P inhibitors and/or their metabolites. Some specific examples include enzymes (e.g. thrombin, factor Xa, COX-2), purified or recombinant CFs, proteins or antibodies (e.g. antithrombin, anti-Xa, anti-CF/P inhibitors), antibody fragments, polypeptides, aptamers, engineered phage (or purified phage tailspike protein), small molecules or other molecular structure capable of binding to, or competing against, CF/P inhibitors or their metabolites. The binding moieties may also compete for one or more analytes against label-conjugated analytes (or analyte analogs) in one example of a competitive assay format. In another example of a competitive assay format, the analytes may compete for binding to a labeled or unlabeled binding moiety (e.g. CF) against a labeled or unlabeled ‘competing moiety’ (e.g. a CF-specific protein such as antithrombin). In another example of a competitive assay format, the analytes may compete for binding to a mobilized labeled binding moiety (e.g. CF-specific antibody) against immobilized analytes (or analyte analogs). In a different assay format, referred to as a non-competitive (or sandwich) format, the binding moieties may also bind and/or capture a complex comprising analyte and a first binding moiety. For example, in the non-competitive format, a first binding moiety may bind selectively to an analyte forming a first complex, which may be subsequently bound to and/or captured by secondary or tertiary binding moieties.

The CF/P inhibitors that can be detected by the present invention include at least direct oral anticoagulant (DOAC) drugs such as rivaroxaban, apixaban, edoxaban, betrixaban, otamixaban, letaxaban, eribaxaban, fondaparinux, dabigatran, argatroban, inogatran, ximelagatran hirudin, lepirudin, desirudin and/or bivalirudin, antiplatelet drugs such as clopidogrel, ticagrelor, and prasugrel, and COX-2 inhibitors such as acetylsalicylic acid, ibuprofen, celecoxib, difenac, etoricoxib, lumiracoxib, and naproxen. This list is not meant to be limiting, and other CF/P inhibitors could be detected by the same process described herein without departing from the scope of the invention. Furthermore, the detection may also involve detection of a metabolite or breakdown byproduct of any of the listed or other CF/P inhibitors.

At step 302, the sample/composition combination is received at an analytical region. The duration of time for the sample/composition combination to be received at the analytical region may be on the order of minutes (e.g. 5-10 minutes), however may be longer or shorter depending on the particular configuration being used. During this time, the one or more analytes in the sample may interact with the at least one binding moiety in the composition and/or the analytical region.

At step 303, a response may be generated indicative of the interaction (or lack thereof) between analytes in the sample and the at least one binding moiety. Depending on the configuration, the response may include the absence of a signal or the generation of a signal. In some embodiments, the absence of a signal may indicate the presence of an analyte. In other embodiments, the absence of a signal may indicate the absence of an analyte. In some embodiments, the presence of a signal may indicate the presence of an analyte. In other embodiments, the presence of a signal may indicate the absence of an analyte. For example, this response may be a signal generated by a label associated with the binding process. Suitable labels for this process include at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal. The amount of signal generated is directly related to the amount of analyte present in the sample and is dependent on the configuration of the test being used. For example, in one configuration, if little or no signal is produced, this indicates that little or no analyte is present in the sample. On the other hand, as the amount of signal being produced increases, this indicates higher amounts of analyte present in the sample. In addition, depending on the strength of the signal produced in step 303, it is possible to estimate an amount of analyte present in the sample. Alternatively, the test may be configured in different format such that the absence of signal or a lower produced signal is indicative of higher amounts of analyte present in the sample, such as in the different configurations and outcomes as discussed in more detail above with respect to FIG. 2.

### ADDITIONAL CONSIDERATIONS

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

1-22 (canceled)

23. A method for detecting, semi-quantifying, or quantifying at least one drug in a patient-derived liquid sample, comprising:
receiving a patient-derived liquid sample by a composition wherein the sample may interact with at least one binding moiety and/or at least one competing moiety, for a sufficient period of time, thereby forming a sample/composition combination capable of generating a signal or response at an analytical region indicative to the presence of said at least one drug in a sample; wherein the at least one drug may be:
one or more of a direct-acting oral anticoagulant (DOAC) drug selected from: rivaroxaban, apixaban, edoxaban, betrixaban, otamixaban, letaxaban, eribaxaban, fondaparinux, dabigatran, argatroban, inogatran, ximelagatran, hirudin, lepirudin, desirudin, and bivalirudin;
and/or one or more of an antiplatelet drug selected from: clopidogrel, ticagrelor, and prasugrel;
and/or one or more of a COX-2 inhibitor drug selected from: acetylsalicylic acid, ibuprofen, celecoxib, difenac, etoricoxib, lumiracoxib, and naproxen;
and/or one or more breakdown product(s) or ‘active’ metabolite(s) deriving from any said drug.

24. The method of claim 23, wherein the patient-derived liquid sample comprises
a patient body fluid such as blood, plasma, serum, urine, saliva or the like;
and/or a sample derived from a patient body fluid;
and/or a sample derived from a patient body fluid subjected to further processing.

25. The method of claim 23, wherein said at least one binding moiety comprises:
(a) antibodies, antibody fragments, or proteins against any drug of claim 23; and/or
(b) engineered phages or purified phage tailspike proteins capable of binding any drug of claim 23; and/or
(c) aptamers or nucleotide sequences, which may be chemically modified or unmodified, capable of binding any drug of claim 23; and/or
(d) purified or recombinant P2Y12 receptor; and/or
(e) enzymes, capable of acting on any drug of claim 23, such as thrombin, factor Xa, and/or cyclooxygenase-2 (COX-2); and/or
(f) any purified or recombinant protein considered to be a ‘salicylic acid binding protein’ (SABP), such as alpha-enolase (ENO-1) or pyruvate kinase isozyme (PKM2), of which approximately 2,000 SABPs have been identified by Hyong Woo Choi et al; and/or
(g) antibodies, antibody fragments, or proteins capable of binding to any drug of claim 23 and forming a mobilized first complex (Drug+Binding Moiety); and/or
(h) antibodies, antibody fragments, or proteins capable of binding to the mobilized first complex of claim 25g and forming a mobilized second complex (First Complex+Second Binding Moiety); and/or
(i) any moiety of claim 25a-h chemically conjugated to a detectable label, fluorophore, probe or nanoparticle reporter comprising at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal.

26. The method of claim 23, wherein the at least one competing moiety comprises
(a) at least one drug of claim 23, or drug analog, which may or may not be physically bound to a protein (e.g. albumin) in order to facilitate its immobilization onto a test region; and/or
(b) antibodies or antibody fragments against COX-2 or any SABP; and/or
(c) antibodies or antigens (such as adenosine diphosphate) that bind selectively to P2Y12; and/or
(d) Antibodies, antibody fragments, proteins (such as antithrombin), or antigens that bind selectively to the at least one binding moiety of claim 25; and/or
(e) engineered phages or purified phage tailspike proteins capable of binding selectively to the at least one binding moiety of claim 25; and/or
(f) aptamers or nucleotide sequences, which may be chemically modified or unmodified, capable of binding selectively to the at least one binding moiety of claim 25; and/or
(g) a chromogenic substrate chemically attached to a detectable moiety, wherein said chromogenic substrate can be selectively cleaved by either COX-2 or any SABP (but not by thrombin or factor Xa) so that the detectable moiety is liberated from the chromogenic substrate thus generating a signal; and/or
(h) any moiety of claim 26a-f chemically conjugated to a detectable label, fluorophore, probe or nanoparticle reporter comprising at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal.

27. The method of claim 23, wherein the at least one drug may be detected all together in a single assay composition or separate in multiple assay compositions.

28. The method of claim 23 wherein after said sufficient time has passed, the detection, semi-quantification, or quantification of the at least one drug is performed by visually inspecting the analytical region of the composition or by using a reader technology to measure the signal at the analytical region of the composition.

29. A test device for detecting, semi-quantifying, or quantifying at least one drug in a patient- derived liquid sample, wherein the format of said test device is a competitive lateral or vertical flow assay.

30. The test device of claim 29, further comprising
(a) a sample application pad for receiving a patient-derived liquid sample;
(b) a conjugate release pad, located downstream relative to the sample application pad along a defined flow path, comprising temporarily immobilized binding or competing moieties which will mobilize when contacted by a liquid sample;
(c) an analytical region, located downstream of the conjugate release pad along the defined flow path, comprising a porous solid material with high protein affinity (I.e. nitrocellulose) allowing for permanent immobilization of one or more test lines comprising binding moieties or competing moieties, which is capable of generating a detectable signal.

31. The test device of claim 29, wherein the at least one drug may be:
one or more of a direct-acting oral anticoagulant (DOAC) drug selected from: rivaroxaban, apixaban, edoxaban, betrixaban, otamixaban, letaxaban, eribaxaban, fondaparinux, dabigatran, argatroban, inogatran, ximelagatran, hirudin, lepirudin, desirudin, and bivalirudin;
and/or one or more of an antiplatelet drug selected from: clopidogrel, ticagrelor, and prasugrel;
and/or one or more of a COX-2 inhibitor drug selected from: acetylsalicylic acid, ibuprofen, celecoxib, difenac, etoricoxib, lumiracoxib, and naproxen;
and/or one or more breakdown product(s) or ‘active’ metabolite(s) deriving from any said drug.

32. The test device of claim 29, wherein the at least one drug(s) may be detected all together in a single assay composition or separate in multiple assay compositions.

33. The test device of claim 29, wherein the binding moieties of claim 30b-c may comprise:
(a) antibodies, antibody fragments, or proteins against any drug of claim 31; and/or
(b) engineered phages or purified phage tailspike proteins capable of binding any drug of claim 31; and/or
(c) aptamers or nucleotide sequences, which may be chemically modified or unmodified, capable of binding any drug of claim 31; and/or
(d) purified or recombinant P2Y12 receptor; and/or
(e) enzymes, capable of acting on any drug of claim 31, such as thrombin, factor Xa, and/or cyclooxygenase-2 (COX-2); and/or
(f) any purified or recombinant protein considered to be a ‘salicylic acid binding protein’ (SABP), such as alpha-enolase (ENO-1) or pyruvate kinase isozyme (PKM2), of which approximately 2,000 SABPs have been identified by Hyong Woo Choi et al; and/or
(g) any moiety of claim 33a-f chemically conjugated to a detectable label, fluorophore, probe or nanoparticle reporter comprising at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal.

34. The test device of claim 29, wherein the competing moieties of claim 30b-c may comprise:
(a) at least one drug of claim 31, or drug analog, which may or may not be physically bound to a protein (e.g. albumin) in order to facilitate its immobilization onto a test region; and/or
(b) antibodies, antibody fragments against COX-2 or any SABP; and/or
(c) antibodies or antigens (such as adenosine diphosphate) that bind selectively to P2Y12; and/or
(d) Antibodies, antibody fragments, proteins (such as antithrombin), or antigens that bind selectively to the binding moieties of claim 33; and/or
(e) engineered phages or purified phage tailspike proteins capable of binding selectively to the binding moieties of claim 33; and/or
(f) aptamers or nucleotide sequences, which may be chemically modified or unmodified, capable of binding selectively to the binding moieties of claim 33; and/or
(g) a chromogenic substrate chemically attached to a detectable moiety, wherein said chromogenic substrate can be selectively cleaved by either COX-2 or any SABP (but not by thrombin or factor Xa) so that the detectable moiety is liberated from the chromogenic substrate thus generating a signal; and/or
(h) any moiety of claim 34a-f chemically conjugated to a detectable label, fluorophore, probe or nanoparticle reporter comprising at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal.

35. The test device of claim 29, wherein the detection, semi-quantification, or quantification of the at least one drug is performed by visually inspecting an analytical region of said test device or by using a reader technology to measure a signal at an analytical region of said test device.

36. A kit comprising the test device of claim 29 and instructions for its use.

37. A test device for detecting, semi-quantifying, or quantifying at least one drug in a patient-derived liquid sample, wherein the format of said test device is a non-competitive lateral or vertical flow assay

38. The test device of claim 37 further comprising
(a) a sample application pad for receiving a patient-derived liquid sample;
(b) a conjugate release pad, located downstream relative to the sample application pad along a defined flow path, comprising at least one temporarily immobilized binding moiety which will mobilize when contacted by a liquid sample;
(c) an analytical region, located downstream of the conjugate release pad along the defined flow path, comprising a porous solid material with high protein affinity (I.e. nitrocellulose) allowing for permanent immobilization of one or more test lines comprising unlabeled binding moieties, which is capable of generating a detectable signal.

39. The test device of claim 37, wherein the at least one drug may be:
one or more of a direct-acting oral anticoagulant (DOAC) drug selected from: rivaroxaban, apixaban, edoxaban, betrixaban, otamixaban, letaxaban, eribaxaban, fondaparinux, dabigatran, argatroban, inogatran, ximelagatran, hirudin, lepirudin, desirudin, and bivalirudin;
and/or one or more of an antiplatelet drug selected from: clopidogrel, ticagrelor, and prasugrel;
and/or one or more of a COX-2 inhibitor drug selected from: acetylsalicylic acid, ibuprofen, celecoxib, difenac, etoricoxib, lumiracoxib, and naproxen;
and/or one or more breakdown product(s) or ‘active’ metabolite(s) deriving from any said drug.

40. The test device of claim 37, wherein the at least one drug(s) may be detected all together in a single assay composition or separate in multiple assay compositions.

41. The test device of claim 37, wherein the at least one temporarily immobilized binding moiety of claim 38b comprises:
(a) antibodies, antibody fragments, or proteins capable of binding to any drug of claim 39 and forming a mobilized first complex (Drug+Binding Moiety); and/or
(b) antibodies, antibody fragments, or proteins capable of binding to the mobilized first complex of claim 41a and forming a mobilized second complex (First Complex+Second Binding Moiety); and/or
(c) enzymes, capable of acting on any drug of claim 39, such as thrombin, factor Xa, and/or cyclooxygenase-2 (COX-2); and/or
(d) purified or recombinant P2Y12 receptor; and/or
(e) any purified or recombinant protein considered to be a ‘salicylic acid binding protein’ (SABP), such as alpha-enolase (ENO-1) or pyruvate kinase isozyme (PKM2), of which approximately 2,000 SABPs have been identified by Hyong Woo Choi et al; and/or
(f) any moiety of claim 41a-e chemically conjugated to a detectable label, fluorophore, probe or nanoparticle reporter comprising at least cellulose nanobeads, colloidal gold, latex beads, europium-containing nanoparticles, quantum dots, surface enhanced Raman scattering (SERS) nanoprobes, plasmonic nanoparticles, upconverting phosphor nanoparticles (UCNPs), fluorescein or other fluorescent dye, or any colorimetric label capable of generating a detectable signal.

42. The test device of claim 37, wherein the unlabeled binding moieties of claim 38c comprise:
immobilized antibodies, antibody fragments, or proteins capable of binding and capturing the mobilized first or second complex of claim 41a-b, but not capable of binding either free drug or free (uncomplexed) binding moiety with any significant or comparable level of affinity.

43. The test device of claim 37, wherein the detection, semi-quantification, or quantification of the at least one drug is performed by visually nspecting an analytical region of said test device or by using a reader technology to measure a signal at an analytical region of said test device.

44. A kit comprising the test device of claim 37 and instructions for its use.
